SEQUENCE LISTING (11 sequences)

SEQ ID NO: 1 - DNA, 63 bp, Artificial Sequence
  Sythetic oligonucleotide
aatgatacggcgaccaccgagatctacactctttccctacacgacgctcttccgatctnnnnn

SEQ ID NO: 2 - DNA, 60 bp, Artificial Sequence
  Sythetic oligonucleotide
nnagatcggaacagcgtcgtgtagggaaagagtgtagatctcggtggtcgccgtatcatt

SEQ ID NO: 3 - DNA, 36 bp, Artificial Sequence
  Sythetic oligonucleotide
caagcagaagacggcatacgagctcttccgatcnnn

SEQ ID NO: 4 - DNA, 33 bp, Artificial Sequence
  Sythetic oligonucleotide
gatcggaagagctcgatatccgtcttctgcttg

SEQ ID NO: 5 - DNA, 25 bp, Artificial Sequence
  Sythetic oligonucleotide
aatgatacggcgaccaccgagatct

SEQ ID NO: 6 - DNA, 33 bp, Artificial Sequence
  Sythetic oligonucleotide
caagcagaagacggcatacgagctcttccgatc

SEQ ID NO: 7 - DNA, 57 bp, Artificial Sequence
  Sythetic oligonucleotide
gatcgaaggagaggacgctgtctgtcgaaggtaaggaacggacgagagaagggagag

SEQ ID NO: 8 - DNA, 57 bp, Artificial Sequence
  Sythetic oligonucleotide
ctaggaaggagaggacgctgtctgtcgaaggtaaggaacggacgagagaagggagag

SEQ ID NO: 9 - DNA, 57 bp, Artificial Sequence
  Sythetic oligonucleotide
aattgaaggagaggacgctgtctgtcgaaggtaaggaacggacgagagaagggagag

SEQ ID NO: 10 - DNA, 55 bp, Artificial Sequence
  Sythetic oligonucleotide
cggaaggagaggacgctgtctgtcgaaggtaaggaacggacgagagaagggagag

SEQ ID NO: 11 - DNA, 53 bp, Artificial Sequence
  Sythetic oligonucleotide
ctctcccttctcgaatcgtaaccgttcgtacgagaatcgctgtcctctccttc

(19) United States
(12) Patent Application Publication
(10) Pub. No.: US 2013/0267428 A1
(43) Pub. Date: Oct. 10, 2013

(54) High throughput digital karyotyping for biome characterization

(71) Applicants: Washington University in St. Louis, St. Louis, MO (US); University of Washington through its Center for Commercialization, Seattle, WA (US)

(72) Inventors: Russell N. Van Gelder, Mercer Island, WA (US); Aaron Lee, St. Louis, MO (US); Valli Muthappan, Baltimore, MD (US)

(21) Appl. No.: 13/764,098

(22) Filed: Feb. 11, 2013

Related U.S. Application Data
(60) Provisional application No. 61/597,516, filed on Feb. 10, 2012.

(51) Int. Cl. C12Q 1/68

(52) U.S. Cl. CPC C12Q 1/6874
USPC 506/4; 506/2; 506/16

(57) ABSTRACT

The invention herein describes a method for identifying a DNA sequence, and oligonucleotide adaptors used in the identification of a DNA sequence.

### RELATED APPLICATIONS

This application is related to U.S. provisional patent application Ser. No. 61/597,516, filed Feb. 10, 2012, the disclosure of which is incorporated by reference.

### FIELD OF THE INVENTION

This invention is related to methods for identifying DNA sequences in a sample.

### BACKGROUND OF THE INVENTION

The human body is a complex biome which includes trillions of individual genomes of thousands of microbial species. Within the body are several characterized microbiomes, including that of the distal gut, vaginal mucosa, oral mucosa, skin, and conjunctiva. While deep sequencing of a complex biome has been performed for characterization of a complex biome, such an approach is not economical or practical for clinical samples, and is very computationally intensive. Human microbiomes have been primarily characterized by 16S ribosomal sequencing for bacterial DNA, and to a lesser extent, by 18S and internal transcribed spacer (ITS) ribosomal sequencing for fungal DNA, but these techniques are not readily adaptable to viruses, phage, or parasites.

### SUMMARY OF THE INVENTION

The invention as disclosed herein provides embodiments for generating and identifying DNA sequences. In one aspect, a method is provided. The method involves receiving sequence-tag data that indicates a first set of sequence tags. Each sequence tag in the first set of sequence tags is associated with a cutting of a nucleic acid sequence by a Type IIB DNA restriction enzyme, and the nucleic acid sequence is associated with one or more unidentified organisms represented in a sample. The method also involves comparing a first sequence tag in the first set of sequence tags to each sequence tag in a second set of sequence tags. Each sequence tag in the second set of sequence tags is associated with a portion of one of a plurality of nucleic acid sequences. The portion is identified based on the Type IIB DNA restriction enzyme, and each nucleic acid sequence in the plurality of nucleic acid sequences is associated with a one or more identified organisms. The method further involves determining identification data that indicates a potential identity of at least one of the one or more identified organisms based on a match between the first sequence tag and a second sequence tag in the second set of sequence tags, and causing a graphical display to provide a visual representation of the identification data.

In another aspect, a device is provided. The device may include a processor and memory having instructions stored thereon to cause the processor to perform functions involving receiving sequence-tag data that indicates a first set of sequence tags. Each sequence tag in the first set of sequence tags is associated with a cutting of a nucleic acid sequence by a Type IIB DNA restriction enzyme, and the nucleic acid sequence is associated with one or more unidentified organisms represented in a sample. The functions also involve comparing a first sequence tag in the first set of sequence tags to each sequence tag in a second set of sequence tags. Each sequence tag in the second set of sequence tags is associated with a portion of one of a plurality of nucleic acid sequences. The portion is identified based on the Type IIB DNA restriction enzyme, and each nucleic acid sequence in the plurality of nucleic acid sequences is associated with a one or more identified organisms. The functions further involve determining identification data that indicates a potential identity of at least one of the one or more identified organisms based on a match between the first sequence tag and a second sequence tag in the second set of sequence tags, and causing a graphical display to provide a visual representation of the identification data.

In yet another aspect, a physical and/or non-transitory computer readable medium is provided. The physical and/or non-transitory computer readable medium may have instructions stored thereon to cause a computing device to perform functions involving receiving sequence-tag data that indicates a first set of sequence tags. Each sequence tag in the first set of sequence tags is associated with a cutting of a nucleic acid sequence by a Type IIB DNA restriction enzyme, and the nucleic acid sequence is associated with one or more unidentified organisms represented in a sample. The functions also involve comparing a first sequence tag in the first set of sequence tags to each sequence tag in a second set of sequence tags. Each sequence tag in the second set of sequence tags is associated with a portion of one of a plurality of nucleic acid sequences. The portion is identified based on the Type IIB DNA restriction enzyme, and each nucleic acid sequence in the plurality of nucleic acid sequences is associated with a one or more identified organisms. The functions further involve determining identification data that indicates a potential identity of at least one of the one or more identified organisms based on a match between the first sequence tag and a second sequence tag in the second set of sequence tags, and causing a graphical display to provide a visual representation of the identification data.

The invention as disclosed herein further provides an oligonucleotide adaptor comprising a nucleic acid structure represented by formula [I]:

5′-L-X-M-N-3′ [I]

wherein:

L is an optional 5′ label;

X is a nucleotide sequence complementary to solid-phase bridge oligonucleotides;

M is an optional nucleotide barcode; and

N is a nucleotide that comprises a sequence capable of hybridizing with a two or three nucleotide 3′ overhang of the Type IIB DNA restriction enzyme.

Specific embodiments of the invention will become evident from the following more detailed description of certain embodiments and the claims.

### BRIEF DESCRIPTION OF THE DRAWINGS

FIG. 1 is a flowchart depicting a method for identifying a DNA sequence, according to some embodiments of the present application.

FIG. 2 provides an example method for generating the first set of sequence tags.

FIG. 3 is a schematic illustrating a conceptual partial view of an example computer program product that includes a computer program for executing a computer process on a computing device, arranged according to at least some embodiments presented herein.

FIG. 4 shows a simplified block diagram depicting example components of an example computing system.

FIG. 5 shows a schematic of the method of the invention.

FIG. 6 is a representative ethidium bromide-stained gel of products using the methods of the invention. Lane 1:1 kb DNA ladder; Lane 2: 100 base pair DNA ladder; Lane 3: PCR amplification of BRISK fragments following ligation of asymmetric adaptors; Lane 4: Amplification of unbound material from biotin column; Lane 5: Amplification of beads following melt and elution of single-stranded DNA; Lane 6: Amplification of material eluted from beads (desired product containing one long and one short adapter); Lane 7: Negative PCR control.

FIG. 7 shows a histogram of tag recovery from an unamplified human blood sample.

FIG. 8 shows observed verses expected recovery of sequence tags by chromosome using method of the invention from human whole blood sample. Closed circles=unamplified DNA; open circles=phi29 amplified DNA.

FIG. 9 shows karyotype analysis of unamplified human blood DNA sample by the method of the invention.

FIG. 10 shows a histogram of tag recovery from an Phi29 amplified sample.

FIG. 11 shows karyotype analysis of amplified human blood DNA sample by the method of the invention.

FIG. 12 shows distribution of genomically unknown sequences in the oral mucosa of three normal human volunteers. PCR primers were designed for each of 8 GUS candidates and performed on salivary samples from three individuals (S1-S3), blood from the individual sequence was originally isolated from (B) and human cell line HEK293 (C). Negative control (NC) contained no template DNA. Universal bacterial 16S rDNA primers were used as positive control for presence of bacterial DNA. Melanopsin positive control contained primers specific for the human opn4 gene sequence and served as control for presence of human DNA. After sequence extension by vectorette-assisted genome walking, GUS 8 was identified as human DNA sequence from clone RP11-318L16 on chromosome 1.

### DETAILED DESCRIPTION OF THE INVENTION

The following description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure.

The invention as disclosed herein provides methods for identifying a DNA sequence, the method comprising: receiving sequence-tag data that indicates a first set of sequence tags, wherein each sequence tag in the first set of sequence tags is associated with a cutting of a nucleic acid sequence by a Type IIB DNA restriction enzyme, wherein the nucleic acid sequence is associated with one or more unidentified organisms represented in a sample; comparing a first sequence tag in the first set of sequence tags to each sequence tag in a second set of sequence tags, wherein each sequence tag in the second set of sequence tags is associated with a portion of one of a plurality of nucleic acid sequences, the portion identified based on the Type IIB DNA restriction enzyme, and wherein each nucleic acid sequence in the plurality of nucleic acid sequences is associated with a one or more identified organisms; determining identification data that indicates a potential identity of at least one of the one or more identified organisms based on a match between the first sequence tag and a second sequence tag in the second set of sequence tags; and causing a graphical display to provide a visual representation of the identification data.

The invention as disclosed provides methods for identifying a DNA sequence, for example biome representational in silico karyotyping (BRISK), which subjects a biome's genomic representation (here designated as a set of sequence tags) generated by a Type IIB restriction endonuclease to massively parallel deep sequencing followed by identification of each DNA sequence tag. Type IIB DNA restriction enzymes cleave both DNA strands at specified locations both upstream and downstream from their recognition sequence, generating a short DNA duplex sequence tag (i.e., restriction fragment). Sequence tags range from 20-40 base pairs in length (depending on the enzyme) with 3′ overhangs at the cut site, and 20-33 base pairs in length for the duplexed portions of the sequence tag. Because the representation of DNA in a set of sequence tags is defined by recognition site of the Type IIB restriction enzyme used, all known human, microbial, viral, fungal, and parasitic sequence tags can be a priori predicted using an in silico virtual digest generating a second set of sequence tags (for example; an in silico virtual digest of the ˜3 billion base pairs of the human genome with the BsaXI Type IIB restriction enzyme results in ˜1.1 million unique sequence tags; and a virtual digestion of bacterial, fungal, plant, and viral sequences yields ˜2.4 million sequence tags).

The method of the invention represents a rapid and highly sensitive method for characterization of complex microbiomes, in addition to being a sensitive means for performing digital karyotyping, such as BRISK. With new sequence information arising from human microbiome research, the utility of this approach will increase. The method of the invention is well suited to analysis of particular microbiomes over time, as analyses are directly comparable from one timepoint to the next; such analysis is currently more efficient and cost-effective than repeated deep sequencing, for example. The methods of the invention are also capable of identifying many known and novel microbial sequences. The method of the invention should find substantial application in the characterization of human and other microbiota.

The method of the invention allows specific amplification of Type IIB endonuclease restriction fragments without cloning, and direct application of these fragments to a massively parallel DNA sequencing platform. The method of the invention may be performed as described on very small amounts of material (on the order of 1 ng starting genomic DNA when phi29 amplification is employed). Furthermore, the method of the invention is quite rapid, requiring ˜6 hours from sample acquisition to initiation of DNA sequencing. Because of the large number of sequence tags generated in the method of the invention, resolution of the digital karyotype approaches the theoretical limit of 4 kb and allows precise mapping of amplifications and deletions.

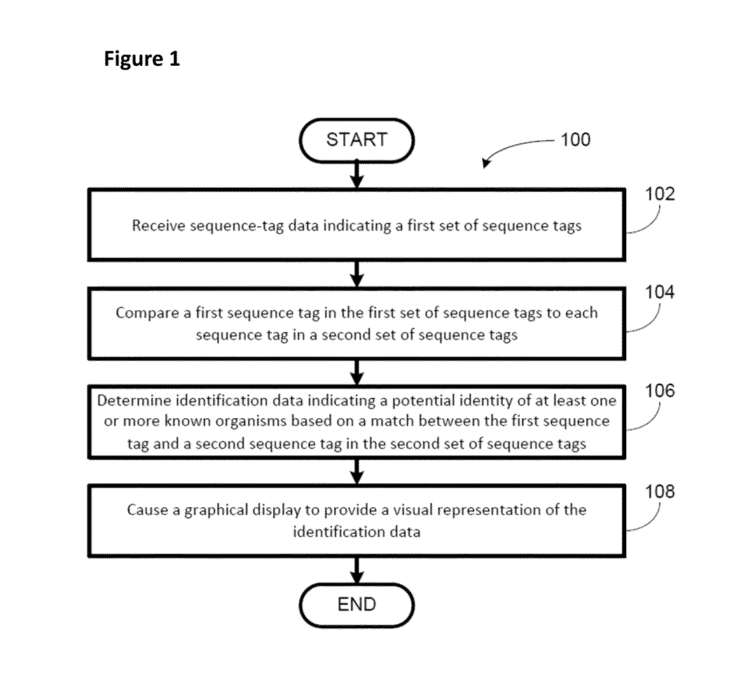

As suggested above, the invention provides methods for identifying a DNA sequence, for example biome representational in silico karyotyping. FIG. 1 is a flowchart depicting a method for identifying a DNA sequence, according to some embodiments of the present application. Method 100 shown in FIG. 1 presents an embodiment of a method that could be performed by a computing device, and may include one or more operations, functions, or actions as illustrated by one or more of blocks 102-108. Although the blocks are illustrated in a sequential order, these blocks may also be performed in parallel, and/or in a different order than those described herein. Also, the various blocks may be combined into fewer blocks, divided into additional blocks, and/or removed based upon the desired implementation. In addition, for the method 100 and other processes and methods disclosed herein, the flowchart shows functionality and operation of one possible implementation of present embodiments. In this regard, each block may represent a module, a segment, or a portion of program code, which includes one or more instructions executable by a processor or electronic circuit for implementing specific logical functions or steps in the process.

The program code may be stored on any type of computer readable medium such as, for example, a storage device including a disk or hard drive. The computer readable medium may include a physical and/or non-transitory computer readable medium, for example, such as computer-readable media that stores data for short periods of time like register memory, processor cache and Random Access Memory (RAM). The computer readable medium may also include physical and/or non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

At block 102, the method 100 may involve receiving sequence-tag data that indicates a first set of sequence tags. In one example, each sequence tag in the first set of sequence tags may be associated with a cutting (or digesting) of a sample containing nucleic acid sequences by a Type IIB DNA restriction enzyme. The nucleic acid sequences may be acquired from a sample that may be represented by a number of different organisms. For instance, a sample of human saliva may include both DNA from a human, and DNA from different bacteria. Some of the organisms, such as the human, may be known or identified, and some of the organisms, such as some of the bacteria, may be unidentified. In some cases, some of the organisms may even be unknown or unidentified. In either case, the nucleic acid sequence may be associated with one or more unidentified organisms represented in the sample.

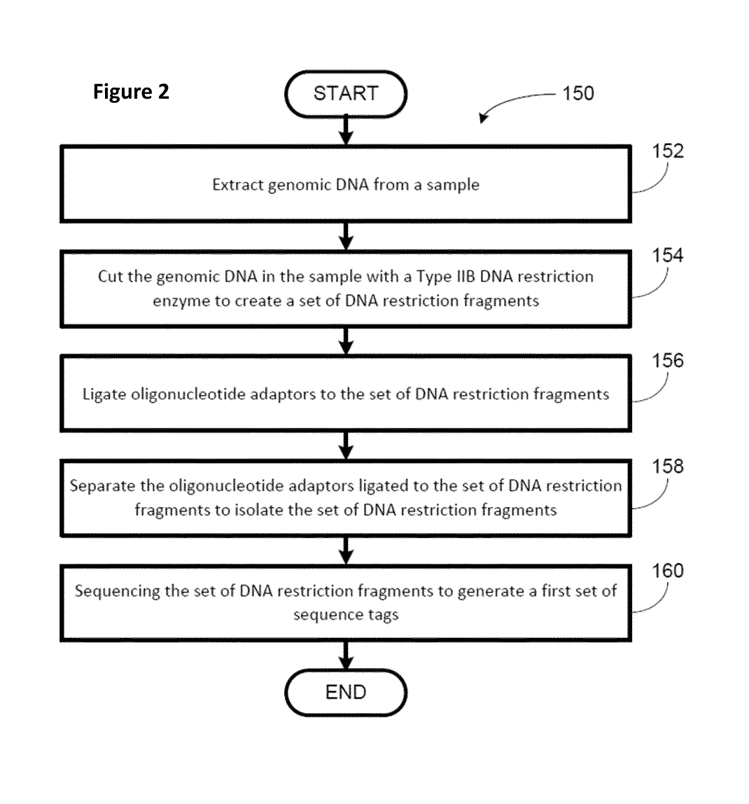

FIG. 2 provides an example method 150 for generating the first set of sequence tags. In one example, the method 150 may be performed before performing the method 100 of FIG. 1. In another example, the method 150 may be performed as part of performing block 102 of FIG. 1. As shown, the method 150 may include blocks 152-160. At block 152, the method 150 may involve extracting genomic DNA from the sample, and at block 154, the method 150 may involve cutting the genomic DNA in the sample with a Type IIB DNA restriction enzyme to create a set of DNA restriction fragments each fragments being approximately 20-33 base pairs in length. In one example, the Type IIB DNA restriction enzyme used for cutting the genomic DNA in the sample may be selected from a group of restriction enzymes including AjuI, AlfI, AloI, ArsI, BaeI, BarI, BcgI, BdaI, BplI, BsaXI, Bsp24I, CjeI, CjePI, CspCI, FalI, HaeI, Hin4I, NgoAVIII, NmeDI, PpiI, PsrI, RdeGBIII, SdeOSI, TstI and UcoMSI.

After the genomic DNA has been cut, block 156 of the method 150 may involve ligating oligonucleotide adaptors to the set of DNA restriction fragments, and block 158 may involve separating the oligonucleotide adaptors ligated to the set of DNA restriction fragments to isolate the set of DNA restriction fragments. At this point, the first set of sequence tags may be generated by sequencing the set of DNA restriction fragments at block 160. As a result of cutting the genomic DNA using the Type IIB DNA restriction enzyme, each sequence tag in the first set of sequence tags may have a length between 20-33 nucleotides. In one example, the first set of sequence tags may be generated to be in a computer-readable format, and included in the sequence-tag data received at block 102.

In one case, the sequence-tag data may be received over a wired or wireless network from a network or local memory storage device. In another case, the sequence-tag data may be entered by a user via a user interface. In either case, the data generated as discussed in FIG. 2 may be received at block 102 using any suitable process or processes.

A set of sequence tags from a DNA sample is determined by the specific Type IIB DNA restriction enzyme used. The sequence tags represent the restriction fragments of the all DNA digested and cut with a particular Type IIB DNA restriction enzyme, and are generated from portions of the DNA sample defined by the specific recognition and cleavage site of the restriction enzyme used. Type IIB DNA restriction enzymes or restriction endonucleases cleave both DNA strands at specified locations both upstream and downstream from their recognition sequence, generating a short DNA duplex sequence tag (i.e., restriction fragment). Sequence tags range from 20-40 base pairs in length (depending on the enzyme) with a 3′ overhangs at the cut site, and 20-33 bp in length for the duplexed portions of the sequence tag. For example, the BsaXI restriction enzyme can be used to generate 27 duplexed base pairs sequence tags with 2-3 base pair 3′ overhangs, for total length of 31-33 base pairs. Additional, non-limiting examples, of Type IIB restriction enzymes include: AjuI, AlfI, AloI, ArsI, BaeI, BarI, BcgI, BdaI, BplI, BsaXI, Bsp24I, CjeI, CjePI, CspCI, FalI, HaeI, Hin4I, NgoAVIII, NmeDI, PpiI, PsrI, RdeGBIII, SdeOSI, TstI and UcoMSI.

The subject matter of the present application as disclosed herein provides for a second set of sequence tags stored in a database, the database further comprising metadata associated with each sequence tag in the second set of sequence tags, wherein metadata associated with the second sequence tag indicates that the second sequence tag is associated with a particular organism of the one or more identified organisms, and wherein the identification data indicates that the potential identity of the at least one of the one or more unidentified organisms comprises the particular organism. Because the representation of DNA in a set of sequence tags is defined by the Type IIB restriction enzyme used, all known human, microbial, viral, fungal, and parasitic sequence tags can be a priori predicted using an in silico virtual digest generating a second set of sequence tags (for example; an in silico virtual digest of the ˜3 billion base pairs of the human genome with the BsaXI restriction enzyme results in ˜1.1 million unique sequence tags; and a virtual digestion of bacterial, fungal, plant, and viral sequences yields ˜2.4 million sequence tags). Bioinformatically, the matching of first and second sets of sequence tags requires only a table-lookup, rather than the computationally intensive DNA alignment methodology required in most deep DNA sequencing techniques (e.g., the analysis avoids performing BLAST for each sequence in the first set of sequence tags against the entirety of sequences in a repository, such as the ˜126,000,000,000 bases in GenBank®). Thus, this second set of sequence tags allows for very rapid bioinformatics analysis (for example complete analysis of samples containing >10⁶ sequence tags can be completed in approximately 15 minutes on a standard desktop personal computer).

Referring back to the method 100 of FIG. 1, block 104 may involve comparing a first sequence tag in the first set of sequence tags to each sequence tag in a second set of sequence tags. The second set of sequence tags may be stored in a database, and retrieved or accessed from the database when performing block 104. In one example, each sequence tag in the second set of sequence tags may be associated with a portion of one of a plurality of nucleic acid sequences. The plurality of nucleic acid sequences may be acquired from a repository of nucleic acid sequences, such as GenBank® from the National Institute of Health. In this case, each of the plurality of nucleic acid sequences acquired may be associated with one or more identified organisms.

In one case, the portion of the one of the plurality of nucleic acid sequences may be identified based on the Type IIB DNA restriction enzyme. For example, each of the plurality of nucleic acid sequences may be cut in silico, or parsed, according to characteristics of the recognition site of the Type IIB DNA restriction enzyme to generate DNA restriction fragments. In other words, DNA restriction fragments may be generated from a computer-simulated digestion and processing of the one of the plurality of nucleic acid sequences according to method 150 of FIG. 2 discussed above. Accordingly, the portion of the one of the plurality of nucleic acid sequences may be identified as a portion corresponding to a generated DNA restriction fragments.

As a result of this process, each sequence tag in the second set of sequence tags may also have a length between 20-33 nucleotides. Further, because the nucleic acid sequences in the plurality of acid sequences acquired from the repository are associated with known or identified organisms, each sequence tag in the second set of sequence tags may be associated with one or more identified organisms. In particular, each sequence tag in the second set of sequence tags may be associated with the same one or more organisms that nucleic acid sequence from which the corresponding sequence tag was cut or parsed.

In one example, the second set of sequence tags may be stored in a database, as indicated above. The database may be a local database stored on a local memory storage device, or a remote database accessed over a wired or wireless network. Also stored in the database may be metadata associated with each sequence tag in the second set of sequence tags. Metadata, as discussed herein, may refer to data descriptive of content, such as other entries or items in the database. For instance, metadata associated with a particular sequence tag may indicate that the particular sequence tag is associated with an organism of the one or more known or identified organisms represented in the repository of nucleic acid sequences. In this case, the metadata may be imported or converted from data from the repository when acquiring the nucleic acid sequences. Furthermore, metadata associated with a particular sequence tag may indicate a genomic location of a sequence tag within an organism.

In one case, the second set of sequence tags in the database may be continually updated with known BsaXI tags by checking for and digesting in silico new or updated sequences that may be available from the repository. For instance, the repository, such as GenBank®, or any other source may be pinged periodically for new or updated sequences. If new or updated sequences are available, the new or updated sequences may be retrieved and processed, as described above to generate sequence tags to be included in an updated second set of sequence tags.

During the comparison between the first sequence tag and each sequence tag of the second set of sequence tags, a match may be found. At block 106, the method 100 may involve determining identification data that indicates a potential identity of at least one of the one or more identified organisms based on a match between the first sequence tag and a second sequence tag in the second set of sequence tags. In one example, if the second sequence tag matching the first sequence tag is associated with a particular organism according to metadata stored in the database, then the identification data may indicate that the potential identity of the at least one of the one or more unidentified organisms from the sample may include the particular organism.

In one case, if the second sequence tag is present only once in a full genome of the particular organism, the metadata associated with the second sequence tag may indicate (i) a genomic location of the second sequence tag and (ii) that the second sequence tag is present only once in the full genome of the particular organism. In another case, if the second sequence tag is associated with only the particular organism and is present two or more times within a genome of the particular organism, the metadata associated with the second sequence tag may indicate that the second sequence tag is a potential identifier of the particular organism. In either case, the information provided by the metadata may then also be included in the identification data.

In a further case, if the second sequence tag is associated with a group of organisms in the one or more identified organisms, including the particular organism, the metadata associated with the second sequence tag may indicate that the second sequence tag is a potential identifier of each organism in the group of organisms. In this case the identification data may indicate that the potential identity of the one or more unidentified organisms may include the organisms in the group of organisms. In one example, the group of organisms may be one of bacteria, fungi, parasites, viruses, phage, vertebrates, or invertebrates.

As stated previously, the sample from which the first set of sequence tags originated may be represented by a number of different organisms. In one example, each sequence tag in the first set of sequence tags may be compared against each sequence tag in the second set of sequence tags. As a result of this comparison, multiple matches between sequence tags from the respective sets of sequence tags may be found. The result of multiple matches may indicate identities (or potential identities) of one or more of the one or more different organisms represented in the sample. Further, depending on the frequency of matches between a sequence tag associated with the particular organism (from the second sequence tag) and sequence tags in the first set of sequence tags, a percentage representation by the particular organism in the sample may be determined. In this case, the percentage representation may further be included in the identification data.

At block 108, the method 100 may involve causing a graphical display to provide a visual representation of the identification data. As discussed previously, the method 100 may be performed by a computing device. In one example, the computing device may further be coupled to the graphical display, which may be a computer monitor. Accordingly, the identification data generated as a result of a match between the first sequence tag and second sequence tag may be provided in the form of the visual representation. A user of the computing device may then review and study the visual representation of the identification data.

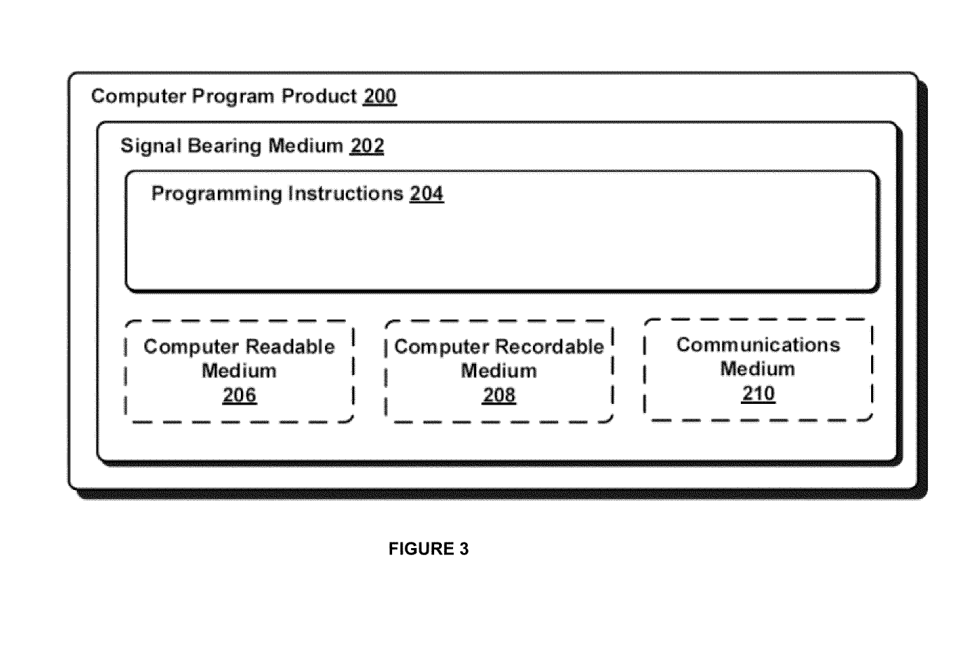

As indicated above, in some embodiments, the disclosed methods may be implemented by computer program instructions encoded on a physical and/or non-transitory computer-readable storage media in a machine-readable format, or on other physical and/or non-transitory media or articles of manufacture. FIG. 3 is a schematic illustrating a conceptual partial view of an example computer program product that includes a computer program for executing a computer process on a computing device, arranged according to at least some embodiments presented herein.

In one embodiment, the example computer program product 200 may be provided using a signal bearing medium 202. The signal bearing medium 202 may include one or more programming instructions 204 that, when executed by one or more processors may provide functionality or portions of the functionality described with respect to method 100 of FIG. 1. In some examples, the signal bearing medium 202 may encompass a physical and/or non-transitory computer-readable medium 206, such as, but not limited to, a hard disk drive, a Compact Disc (CD), a Digital Video Disk (DVD), a digital tape, memory, etc. In some implementations, the signal bearing medium 102 may encompass a computer recordable medium 208, such as, but not limited to, memory, read/write (R/W) CDs, R/W DVDs, etc. In some implementations, the signal bearing medium 202 may encompass a communications medium 210, such as, but not limited to, a digital and/or an analog communication medium (e.g., a fiber optic cable, a waveguide, a wired communications link, a wireless communication link, etc.). Thus, for example, the signal bearing medium 102 may be conveyed by a wireless form of the communications medium 110.

The one or more programming instructions 204 may be, for example, computer executable and/or logic implemented instructions. In some examples, a processing unit may be configured to provide various operations, functions, or actions in response to the programming instructions 204 conveyed to the processing unit by one or more of the computer readable medium 206, the computer recordable medium 208, and/or the communications medium 210.

The physical and/or non-transitory computer readable medium could also be distributed among multiple data storage elements, which could be remotely located from each other. The computing device that executes some or all of the stored instructions could be a computing device such as any of those described above. Alternatively, the computing device that executes some or all of the stored instructions could be another computing device, such as a server.

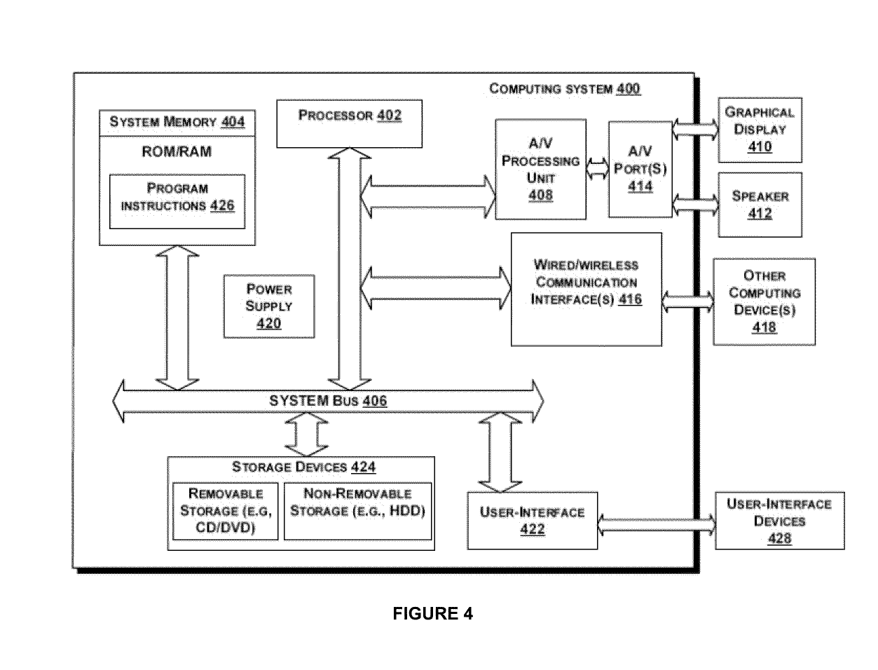

FIG. 4 shows a simplified block diagram depicting example components of an example computing system 400, which may be implemented as a computing device or server associated with the computer product of FIG. 3. Computing system 400 may include at least one processor 402 and system memory 404. In an example embodiment, computing system 400 may include a system bus 406 that communicatively connects processor 402 and system memory 404, as well as other components of computing system 400. Depending on the desired configuration, processor 402 can be any type of processor including, but not limited to, a microprocessor (μP), a microcontroller (μC), a digital signal processor (DSP), or any combination thereof. Furthermore, system memory 404 can be of any type of memory now known or later developed including but not limited to volatile memory (such as RAM), non-volatile memory (such as ROM, flash memory, etc.) or any combination thereof.

The example computing system 400 may include various other components as well. For example, the computing system 400 may include an A/V processing unit 408 for controlling graphical display 410 and speaker 412 (via A/V port 1014), one or more communication interfaces 416 for connecting to other computing devices 418, and a power supply 420. Graphical display 410 may be arranged to provide a visual depiction of various input regions provided by user-interface module 422. User-interface module 422 may be further configured to receive data from and transmit data to (or be otherwise compatible with) one or more user-interface devices 428.

Furthermore, the computing system 400 may also include one or more data storage devices 424, which can be removable storage devices, non-removable storage devices, or a combination thereof. Examples of removable storage devices and non-removable storage devices include magnetic disk devices such as flexible disk drives and hard-disk drives (HDD), optical disk drives such as compact disk (CD) drives or digital versatile disk (DVD) drives, solid state drives (SSD), and/or any other storage device now known or later developed. Computer storage media can include volatile and nonvolatile, removable and non-removable media implemented in any method or technology for storage of information, such as computer readable instructions, data structures, program modules, or other data. For example, computer storage media may take the form of RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium now known or later developed that can be used to store the desired information and which can be accessed by computing system 400.

According to an example embodiment, the computing system 400 may include program instructions 426 that are stored in system memory 404 (and/or possibly in another data-storage medium) and executable by processor 402 to facilitate the various functions described herein including, but not limited to, those functions described with respect to FIGS. 1 and 2 for identifying a DNA sequence. Although various components of computing system 400 are shown as distributed components, it should be understood that any of such components may be physically integrated and/or distributed according to the desired configuration of the computing system.

The invention as disclosed herein further provides an oligonucleotide adaptor comprising a nucleic acid structure represented by formula [I]: 5′-L-X-M-N-3′ [I], wherein: L is an optional 5′ label; X is a nucleotide sequence complementary to solid-phase bridge oligonucleotides; M is an optional nucleotide barcode; and N is a nucleotide that comprises a sequence capable of hybridizing with a 3′ overhang of the Type IIB DNA restriction enzyme.

The optional label (L) of the oligonucleotide adaptor as used herein refers to any linked molecule or affinity based sequence fused, at any position (typically the 5′ or 3′ end) of a nucleotide sequence. The presence of a suitable label may serve to improve detection, purification or other characteristics of the oligonucleotide. Suitable affinity based labels include any sequence that may be specifically bound to another moiety (non-limiting examples include biotin, poly-histidine, Myc, FLAG, HA, glutathione-S-transferase or a magnetic bead). In some instances, a linked molecule may be a light emitting reporter that may include any domain that can report the presence of an oligonucleotide. Suitable light emitting reporter domains include luciferase, fluorescent proteins or light emitting variants thereof.

A nucleotide sequence complementary to solid-phase bridge oligonucleotides (X) allows for cloning-free DNA amplification and next generation sequencing by attaching single-stranded DNA fragments to a solid surface known as a flow cell, and conducting solid-phase bridge amplification of single-molecule DNA templates. In this process, one end of single DNA molecule is attached to a solid surface using a nucleotide sequence complementary to solid-phase bridge oligonucleotides; the molecules subsequently bend over and hybridize to complementary adapters (creating the “bridge”), thereby forming the template for the synthesis of their complementary strands. After amplification, a flow cell with more than 40 million clusters is produced, wherein each cluster is composed of approximately 1000 clonal copies of a single template molecule. The templates are sequenced in a massively parallel fashion using a DNA sequencing-by-synthesis approach. The Illumina/Solexa approach is one example of next generation sequence that employs this method.

An optional nucleotide barcode (M) as used herein refers to pre-determined bases potentially used as barcode for multiplex sequencing. A barcode may comprise 2, 3, 4 or more pre-determined nucleotide bases that serve as a unique identifier or index that allows for the identification of a particular sample within a pool of samples. Pooling samples into a single lane of a flow cell in next generation sequencing exponentially increases the number of samples analyzed in a single run without drastically increasing cost or time. The optional barcodes of the method of the invention comprise a 2 Levenshtein edit distance difference in order to be certain samples within a pool would not be mixed by any errors potentially introduced during the sequencing process, as a result, a 2 bp barcode usually allows for 16 samples, but a 2 ED difference limits this to 4 samples. A 3 bp barcode with a 2 ED allows for 16 unique barcodes within a pool; and a 4 bp with a 2 ED allows for 64 unique barcodes within a pool.

The (N) of the oligonucleotide adaptor comprises a sequence capable of hybridizing with the random nucleotide 3′ overhang of the restriction enzyme cut site. As previously described, Type IIB DNA restriction enzymes generate sequence tags ranging in length from 20-40 base pairs and (N) comprises a plurality nucleotide sequences complementary to the random 3′ overhangs at the cut sites on both sides of the recognition site, depending on the Type IIB enzyme the 3′ overhangs may be 2-6 base pairs.

Aspects of this disclosure are directed to a method for complete characterization of a defined representation of all DNA contained within a sample. In an embodiment, the method can characterize the host genomic DNA within the sample (e.g., generate an in silico karyotype). In another embodiment, the method can identify known and unknown non-host DNA in a sample (e.g., commensal or pathogenic DNA).

In some embodiments, the method can include extraction of DNA from a target and digestion of the DNA with an enzyme (e.g., Type IIB restriction endonuclease). The method can also include ligation of DNA adaptors to digested DNA. In another embodiment, the ligation step includes the ligation of two different adaptors to the digested DNA. The method can further include selection of DNA selectively ligated with (two) different adaptor sequences (e.g., one can be biotinylated). The method can also include amplification of the selected DNA by polymerase chain reaction (PCR), and high throughput DNA sequencing on, for example, a massively parallel platform, for example Illumina/Solexa platform. The method can further include bioinformatic parsing of the resulting sequence tags, e.g., for rapid mapping of sequences to host chromosome (e.g., human), and identification of unknown sequences (for example, matching a sequence tag from a sample to the ˜1.1 million sequence tags in the human genome rather than aligning each tag to the ˜3 billion base pairs of the human genome). In yet another embodiment, the identification of unknown sequence tags can include mapping the sequence tag to a known sequence database, such as a known microbial sequence database. In another embodiment, the method can also include aligning, combining or “walking from” obtained sequence tags to obtain larger stretches of DNA of an unknown or unidentified organism.

Some examples disclosed herein characterize the Type IIB restriction enzyme BsaXI, specific biotinylated and non-biotinylated oligonucleotide primers, and the Solexa/Illumina parallel sequencing platform. However, one of ordinary skill in the art will recognize that the disclosure is not limited to these aspects and these are described herein only as examples. Accordingly, the disclosure should be read as to broadly incorporate the use of other suitable features and compositions for accomplishing the method.

### EXAMPLES

### Methods

### Subjects

DNA was collected from venous blood and buccal swabs of healthy volunteers. This study was performed with informed consent, under Institutional Review Board approval of Washington University Medical School and University of Washington Medical School.

### Preparation of Genomic DNA

Genomic DNA (gDNA) was extracted from the 293T cell line (ATCC, CRL-11268) and E. coli (Invitrogen) using the DNEasy Blood and Tissue kit (Qiagen). Human blood genomic DNA (gDNA) was extracted using the Paxgene kit (Qiagen), and gDNA of microbiome from buccal brushings were harvested using the Purgene C kit (Qiagen). The gDNA was eluted into deionized, distilled water (ddH2O). 3 ug gDNA was used for each analysis.

BsaXI Digest of gDNA

After extraction, the gDNA was digested using a Type IIB restriction endonuclease, BsaXI (New England Biolabs), using manufacturer's recommended buffer and reaction conditions at 37 degrees Celsius for 16 hours. Cleaving of all genomic DNA within a microbiome sample with the restriction enzyme generates a portion of all the gDNA of a microbiome sample—i.e., a set of sequence tags.

### Preparation of Adaptors

Adaptors complementary to the solid-phase bridge oligonucleotides on the Illumina Genome Analyzer's flow cell were synthesized and purified by high performance liquid chromatography (Integrated DNA Technologies). The longer adaptor was:

(SEQ ID NO: 1)
5′-AATGATACGGCGACCACCGAGATCTACACTCTTTCCCTACACGACG
CTCTTCCGATCTMMNNN-3′,

where the MM comprises two pre-determined bases (AA, TT, CC, GG) potentially used as barcode for multiplex sequencing, and NNN comprises a sequence capable of hybridizing with a two or 3 base pair 3′ overhang of the restriction fragment. The complement for this adaptor was:

(SEQ ID NO: 2)
5′-MMAGATCGGAACAGCGTCGTGTAGGGAAAGAGTGTAGATCTCGGTG
GTCGCCGTATCATT-3′.

The shorter tag was biotinylated (biotin):

(SEQ ID NO: 3)
5′-Biotin-CAAGCAGAAGACGGCATACGAGCTCTTCCGATCNNN-3′.

The complement to short biotinylated adaptor was:

(SEQ ID NO: 4)
5′-GATCGGAAGAGCTCGATATCCGTCTTCTGCTTG-3′

The adaptors were reconstituted in ddH2O to create a 10 mM solution. The adaptors were annealed by placing the equimolar mix in a boiling water bath for two minutes, then removing the bath from the heat source and allowing to cool to room temperature for approximately 3 hours. The double stranded adaptors were diluted in 1×TE to a working solution of 1 mM.

### Ligation of Adaptors to BsaXI Restriction Fragments

Restriction fragments representing all the gDNA of a sample were ligated to the adaptors using T4 DNA ligase (New England Biolabs) under standard conditions, modified by additional ATP (Sigma-Aldrich) at 1 uM. Ligation was carried out at 4 degrees Celsius for 1 hour.

### Separation of Products on a Biotin-Streptavidin Column

The restriction fragments ligated to the adaptors were separated on a Dynabead column (Invitrogen) using magnetic stand (Invitrogen) to isolate the asymmetric ligation product of interest. First, the beads were washed twice with 2× binding and wash buffer (10 mM Tris-HCl at pH 7.5; 1 mM EDTA; 2M NaCl). The beads were resuspended in a half-volume of 2× binding and wash buffer, and the restriction fragments ligated to the adaptors were added to the column. After shaking on a horizontal rotator for 20 minutes, the supernatant was removed, and the beads were washed twice with 1× binding and wash buffer.

### Nick-Translation Using Bst DNA Polymerase

Bound products were incubated with 0.4 mM dNTPs (Sigma) and Bst DNA polymerase (New England Biolabs) under manufacturer's recommended conditions. After shaking at 65 degrees Celsius for 20 minutes, the supernatant was removed and the beads were washed twice with 1× binding and wash buffer.

Collect ssDNA Library Containing Asymmetric Product of Interest

To remove the product of interest (i.e. 33 bp restriction fragment tag with one short and one long adaptor ligated), single stranded DNA (ssDNA) was melted from the column using a solution of 100 mM NaCl and 125 mM NaOH. After addition of the melt solution, the column was shaken on a vertical rotator for 10 minutes. The supernatant was removed on the magnet and neutralized using an equal volume of a neutralization solution made of buffer PBI from the Qiaquick PCR purification kit (Qiagen) and 0.15% acetic acid.

PCR Amplification of ssDNA Library

To amplify the restriction fragment tags, a PCR using Phusion Taq (Finnzymes) was performed. The sequence of the 5′ primer for this reaction was:

(SEQ ID NO: 5)
5′-AATGATACGGCGACCACCGAGATCT-3′;

the sequence of the 3′ primer for this reaction was:

(SEQ ID NO: 6)
5′-CAAGCAGAAGACGGCATACGAGCTCTTCCGATC-3′.

The PCR was performed using a rapid cycling method with 25 cycles of: 94 degrees Celsius for 30 seconds and 72 degrees Celsius for 15 seconds. To prepare samples for high throughput sequencing, ten identical PCR products were combined and purified using the Qiaquick PCR purification kit (Qiagen).

### Bioinformatic Analysis of Sequencing Results

All available human and microbial genomes from National Center for Biotechnology Information (NCBI) were initially downloaded in February 2007, and updated daily since that time. The downloaded DNA was then virtually digested with the BsaXI restriction enzyme cleavage site to produce a set of sequence tags 33 base pairs in length mapped to their respective sources and locations. To analyze the sequencing information, raw sequences that matched the restriction enzyme site were identified and only sequence tags that appeared more than once were analyzed. The 27 base pairs surrounding the BsaXI recognition sequence was used for analysis. The resulting sequence tags were filtered against the library of sequence tags from the human genome by finding the shortest edit distance (ED) from each sample sequence tag to the library sequence tag. Based upon an empirically-derived, distribution-based analysis, a cutoff of 3 ED was used to classify a tag as a match to the human genome. All remaining sequence tags were similarly matched against all sequenced bacterial, viral, and fungal genomes that were present in the non-redundant NCBI database. Individual sequence tags that were 3 ED from the nearest known genomes were classified as a ‘genomically unknown sequence’ (GUS). GUS tags were then Basic Local Alignment Search Tool (BLAST) searched against the entire NCBI non-redundant database. For sequence tags matching sequences in the microbial database, analysis was performed at the level of genus, as many subspecies of particular microbial genera had identical sequence tags.

The frequency of the sequence tag in the sample (observed) was divided by the frequency of the sequence tag in the virtually digested human genome (expected); this value was rounded to the nearest whole number to create a score for each organism in the sample. For in silico karyotyping, single-frequency human library sequence tags unique to each chromosome were identified. Chromosome distribution maps were generated by dividing observed sequence tag density over expected tag density per contiguous 1000 unique tags.

### Genome-Walking Protocol to Extend GUS Tags

A vectorette protocol (Ko et al. 2003) was used to find adjacent sequence to GUS tags. Vectorette libraries of phi29 amplified buccal mucosal DNA from the original sample were constructed using eight restriction enzymes (BglII, BclI, BstBI, BsaHI, XbaI, SpeI, MfeI, EcoRI; New England Biolabs). The restriction products were ligated to vectorette adaptors annealed to an imperfect complement that created a bubble structure in each adaptors. The four types of vectorette adaptors were complementary to the four types of overhangs created by the restriction enzymes. The sequences for the four vectorette adaptors were:

Vect 57 GATC:
(SEQ ID NO: 7)
5′-GATCGAAGGAGAGGACGCTGTCTGTCGAAGGTAAGGAACGGACGAGAGAAGGGAGAG-3′;
Vect 57 CTAG:
(SEQ ID NO: 8)
5′-CTAGGAAGGAGAGGACGCTGTCTGTCGAAGGTAAGGAACGGACGAGAGAAGGGAGAG-3′;
Vect 57 TTAA
(SEQ ID NO: 9)
5′-AATTGAAGGAGAGGACGCTGTCTGTCGAAGGTAAGGAACGGACGAGAGAAGGGAGAG-3′;
Vect 55 GC
(SEQ ID NO: 10)
5′-CGGAAGGAGAGGACGCTGTCTGTCGAAGGTAAGGAACGGACGAGAGAAGGGAGAG-3′
The sequence for the mismatched complement was:
Vect 53:
(SEQ ID NO: 11)
5′-CTCTCCCTTCTCGAATCGTAACCGTTCGTACGAGAATCGCTGTCCTCTCCTTC-3′.

Before ligation, the adaptors were mixed with the restriction fragments at a final concentration of 0.02 uM and incubated at 65 degrees Celsius for 5 minutes. To ensure optimal annealing, the block containing samples was removed from the heat source and allowed to cool to room temperature, and then placed at 4 degrees Celsius for 1 hour. Subsequently, the T4 DNA ligase (New England Biolabs), T4 DNA Ligase buffer (New England Biolabs) and 10 uM ATP (Sigma-Aldrich) were added and the reaction was incubated at 16 degrees Celsius overnight.

After construction, the DNA library was used for PCR with primers to the unique GUS tag and primers to the vectorette adaptors at a final concentration of 0.25 uM. HotStarTaq (Qiagen) was used under standard conditions in a step-down PCR. Three samples of each DNA digest in the library were run at a low, medium and high temperature during each anneal step to determine if bands were true products or secondary to PCR artifacts. The temperature conditions for the PCR were 95 degrees Celsius for 14 minutes; denaturing at 95 degrees Celsius for 1 minute, annealing across a gradient of 63 to 72 degree Celsius gradient for 1 minute, extension at 72 degrees Celsius for 2 minutes for 5 cycles; denaturing at 95 degrees Celsius for 1 minute, annealing across a gradient of 59 to 68 degrees Celsius for 1 minute, then extension at 72 degrees Celsius for 2 minutes for 5 cycles; denaturation at 95 degrees Celsius for 45 seconds, annealing across a gradient of 55 to 64 degrees Celsius for 1 minute, then extension at 72 degrees Celsius for 2 minutes for 10 cycles; denaturing at 95 degrees Celsius for 45 seconds; then annealing across a gradient for 51 to 60 degrees Celsius for 1 minute, then extension at 72 degrees Celsius for 2 minutes for 10 cycles; final extension was done at 72 degrees Celsius for 10 minutes.

Products from the PCR were separated on a 2% Tris-Acetate-EDTA agarose gel and bands appearing across all annealing temperatures for a particular set of DNA in the library were extracted using the DNA Clean and Concentrator (Zymo Research). These products were transformed and cloned using the Topo TA pCR 2.1 kit (Invitrogen). Cloned plasmids were extracted using the Qiaprep Spin Miniprep Kit (Qiagen) and the DNA was subjected to standard dye-terminator sequencing.

Confirmation of Sequences Obtained from Genome Walking

To confirm that sequences extracted by genome walking were present in the sample, PCR primers were designed outside the original tag sequence and used to amplify the initial DNA sample. The PCR used Fisher Bioreagents Taq DNA polymerase (Fisher) under standard conditions. The temperature conditions for the PCR were 94 degrees Celsius for 2 minutes; denaturing at 94 degrees Celsius for 30 seconds, annealing at a temperature determined by primer melting temperature (Tm) for 30 seconds, and extension at 72 degrees Celsius for 30 seconds for 20 cycles, and then a final extension at 72 degrees Celsius for 5 minutes.

### Accession Numbers

NCBI accession numbers for GUS sequences are: gb|FI185049.1 gb|FI185051.1 gb|FI185052.1 gb|FI185053.1 gb|FI185054.1 gb|FI185056

### Overview of the Subject Matter of Present Application

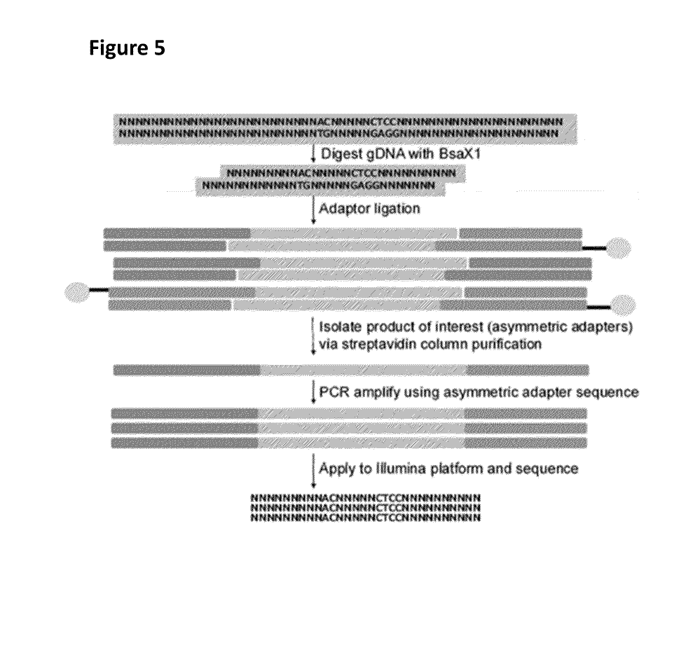

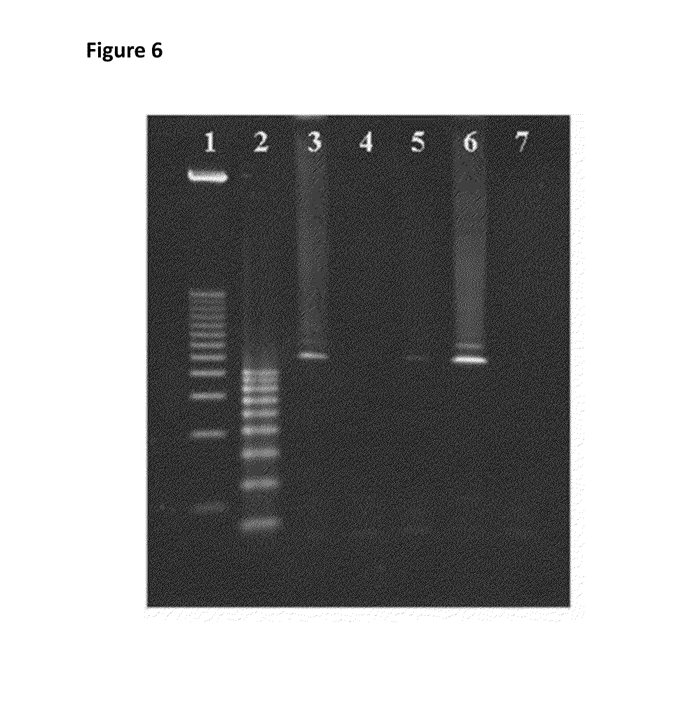

A schematic of the subject matter of present application is shown in FIG. 5. A Type IIB restriction endonuclease (BsaXI) with a 6 base pair (bp) recognition sequencing yielding a 33 bp restriction fragment (i.e. a 27 bp double stranded sequence tag with two 3 bp single-stranded overhangs) was used to generate the representation. Asymmetric adaptor sequences designed to interface directly with the Illumina high throughput sequencing method were ligated to the digested DNA; one adaptor was additionally biotinylated on the 5′ end. The ligation products were bound to a streptavidin column, gaps were repaired with a nicktranslating DNA polymerase, and the desired products (those having different adaptors on each end) were melted off the column and captured. Following polymerase chain reaction-mediated amplification, the representation was directly applied to the Illumina sequencing platform (FIG. 6 is a representative agar gel of the products produced by digesting all DNA is a sample with BsaXI).

After sequencing, 27 bp of each sequence tag (the double stranded portion of the representation) was parsed and matched against a database containing all tags resulting from a virtual BsaXI digest of all sequences from GenBank® divisions of primates, bacteria, invertebrates, fungi, plants, phages, and viruses (GenBank® Release 178.0). In silico digestion of the reference human genome with BsaXI yielded 1.3 million fragments of which 1.1 million were unique sequences. Sequence tags matching human DNA were mapped to position forming a karyotype with ˜4 kb resolution. Virtual digestion of bacterial, fungal, plant, and viral sequences yielded 2.4 million sequence tags. Of these, only 418 tags (0.02%) were found in both human and microbial, fungal, or viral databases. These tags were not used for assignment.

Matches to microbial and viral sequences were then tallied. Microbial and viral tags were assorted in the database to two categories: unique, and ambiguous. A unique tag was found only in a single species. Ambiguous tags were found in more than one organism (for instance, between two or more species of one genus). Of the 1.7 million tags in the bacterial and viral dataset, 1.2 million were unique (68.6%). A ‘unique’ score for each microbial or viral species was calculated based on the number of sequenced tags that were unique matches for that organism. A global score was calculated for each species as well, which is a sum of the unique score and a fractional score for each ambiguous tag (for instance, a tag appearing once matching five species would weight 0.2 for any specific species). Scores were generated for each microbe or virus. To be assigned as ‘present’, an empirical criterion of recovery of at least two, independent, unique tags for that organism was applied.

To analyze the remaining (unmatched) tags, a Levenshtein edit distance model was employed (Yujian and Bo 2007). Empirical analysis of human and microbial tags within the database reveals that fewer than 0.086% of human tags were within 3 Levenshtein edit distances (e.g., single base changes, additions, or deletions) of the nearest microbial 27-mer tag. The average human sequence is 6.5 edit distances from the closest microbial tag. Tags greater than 3 edit distances from nearest human match, but not matching any tags in the microbial or viral databases, were taken to represent potentially novel sequences and were subjected to further analysis.

In some embodiments, the bioinformatic analysis of sequence data includes one or more of the following steps:
1. BsaX1 cut sites were identified from the Illumina sequencing file.
2. Tags were then matched against a database populated by GenBank® cut sites.
3. If no cut sites were identified (0 edit distance) then 1 base permutator was applied and matched against the database (finding tags within 1 edit distance).
4. If any tags were identified as human, mouse, or rat or if they match against a primate or rodent organism then they were sorted into a different file.
5. If any tags were identified as bacterial or viral then they were sorted into another file.
6. Digital karyotypes were then derived from the human, mouse, and rat reference genomes.
7. Samples from cases and controls were compared against each other using a permutation bootstrap heuristic to identify statistical outliers that were differentially expressed in either at the tag level or at the organism level.

### Example 1

### Application to Digital Karyotyping

The digital karyotyping capabilities of the method of the invention were initially characterized by analyzing the digital karyotype of an aseptically acquired human blood sample. Starting from 3 ug of genomic DNA, a total of 12,529,752 tags were identified from the human blood sample. Of these, 11,844,721 (95%) were perfect matches to tags in the human database. Of the 324,592 non-matching distinct tags, 44,785 were found in other aseptically-obtained human blood or human cell line samples, suggesting these are polymorphic or undocumented human sequences. An additional 199,016 tags were within 3 Levenshtein edit distances of nearest human match, again suggesting either polymorphic human sequence or amplification or sequencing error. Thus able 99.36% of tags from the human blood sample were assigned to human origin. The origin of the remaining tags was not known but may represent additional, individual polymorphism as has recently been described for human Alu sequences (Hormozdiari et al. 2010). Estimation of sequencing error was accomplished by analyzing known, single frequency human BsaX1 sites and comparing recovered tags from an aseptically obtained human blood sample to reference human sequences. Levenshtein edit distance for each recovered tag from the reference tag was calculated, and the mode frequency for each known single frequency site was considered as sample normative to account for polymorphisms. Deviations from normative frequency were then calculated and averaged across all sites. Based on this analysis it was estimated that sequencing error accounts for <1% of assignment of non-human tags. In total, 78.8% of all predicted human tags were recovered. Each predicted tag was recovered on average 5.51 times.

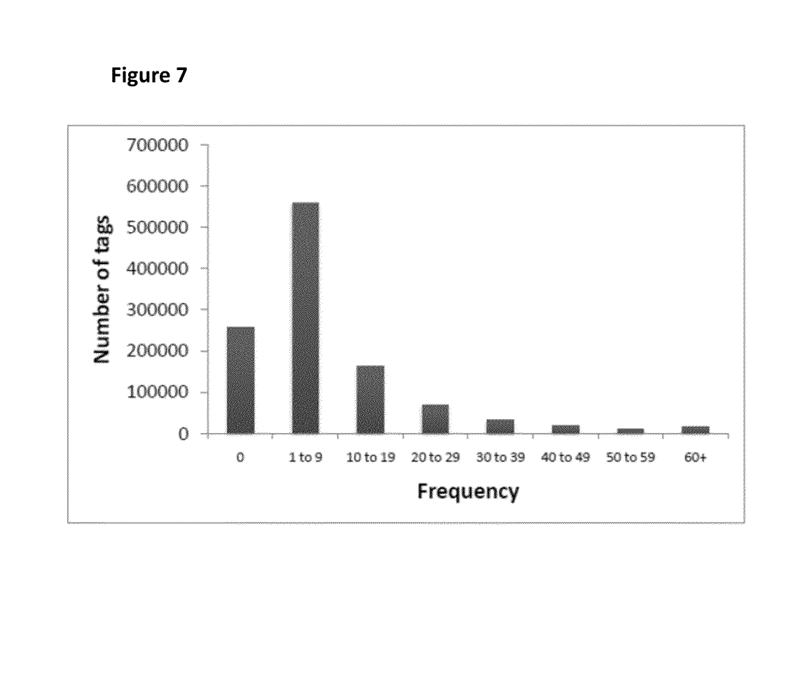

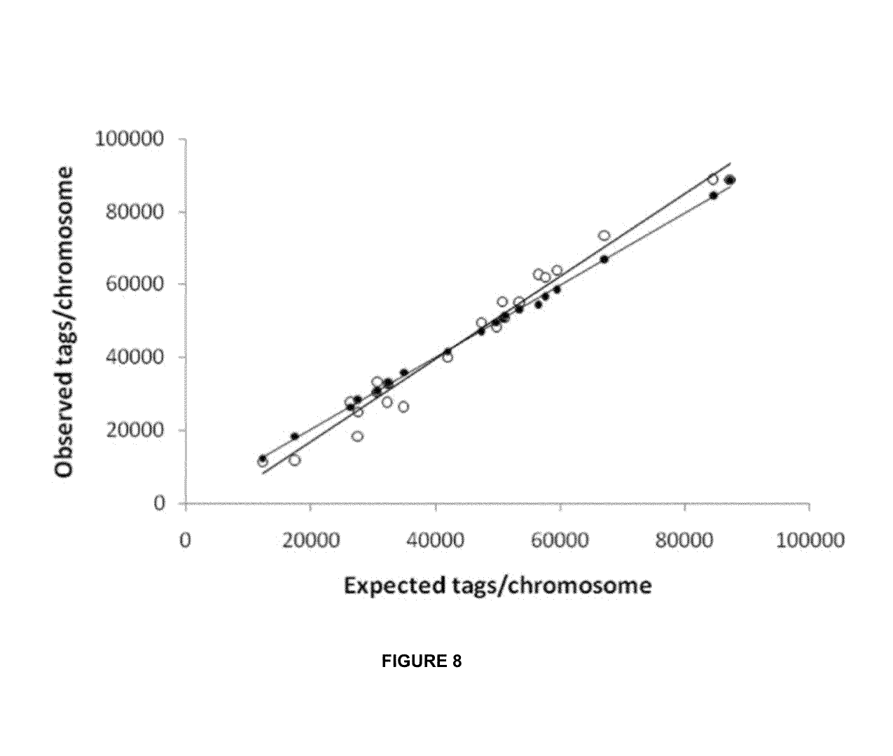

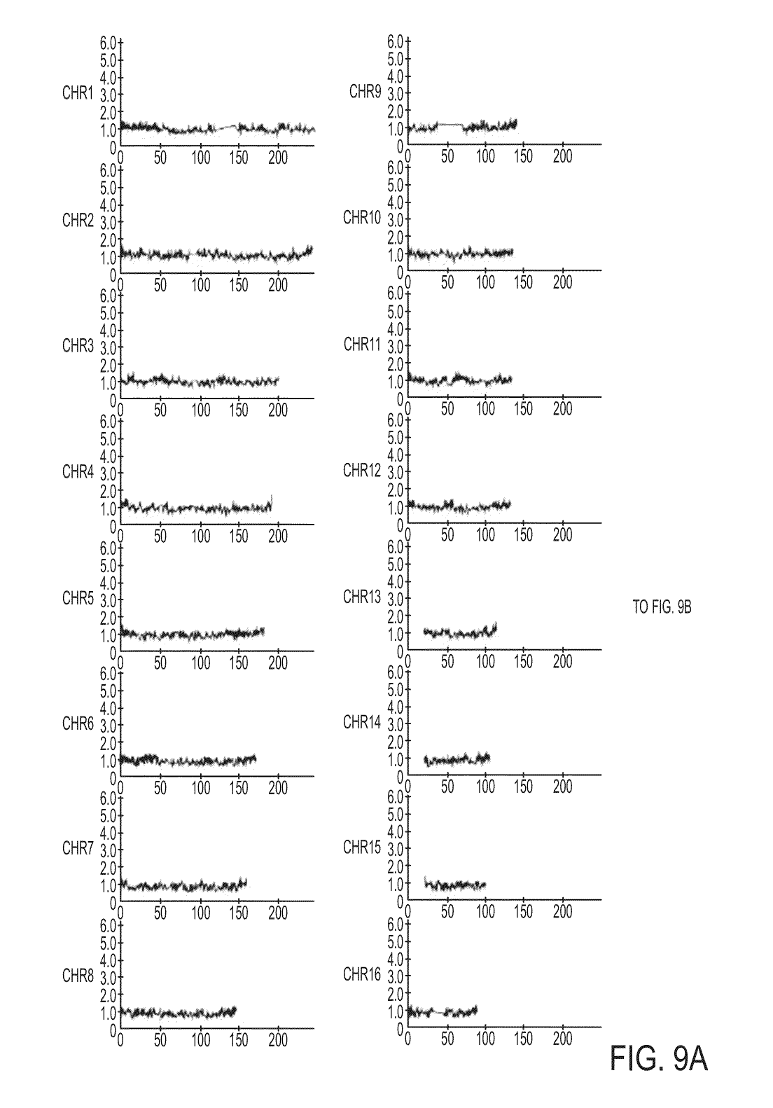

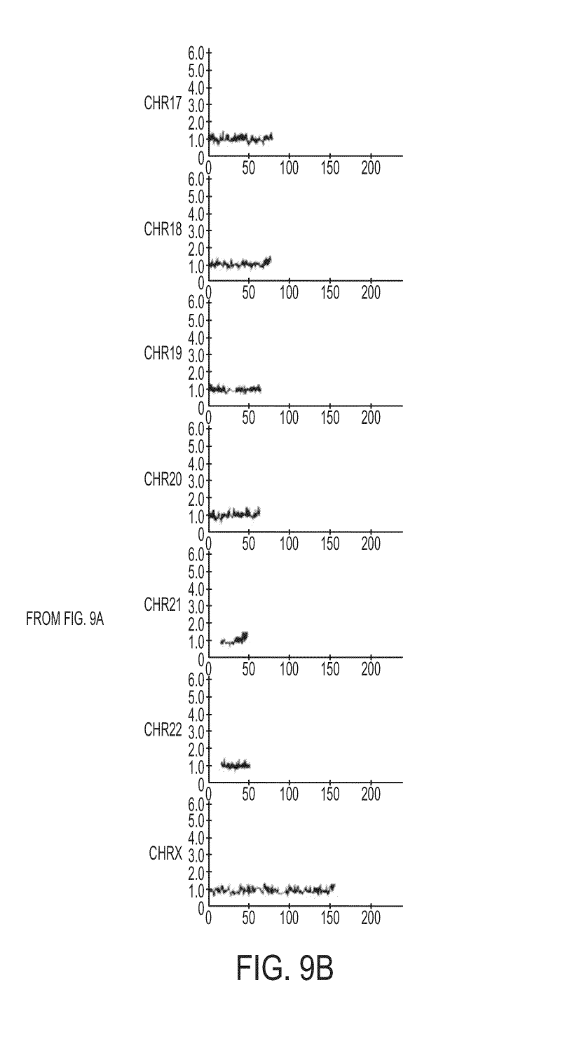

The distribution of quantitative tag recovery for single-frequency tags is shown in FIG. 7. Comparison of number of observed tags vs. expected tags by chromosome revealed very high correlation (Table 1 and FIG. 8), r²=0.999). Mapping of individual tags to chromosome locations revealed a normal XY karyotype (FIG. 9). No tags met criteria for match to microbial sequence. Eight tags were found to match viral sequences: six tags unique for human endogenous retrovirus H, and two tags unique for human endogenous retrovirus K.

Table 1 shows BsaXI tag recovery by experiment

| | Phi29 | Total sequence | Human | Microbial | |
|---|---|---|---|---|---|
| Sample | amplified | tags | matches | matches | Unknown |
| Human blood | No | 12,529,752 | 11,844,721 | 8 (viral) | 685,023 |
| | | | (95%) | (0%) | (5%) |
| Human blood | Yes | 4,091,327 | 3,868,735 | 3 (viral) | 222,589 |
| (Phi29 amplified) | | | (95%) | (0%) | (5%) |
| Buccal Sample 1 | Yes | 3,400,930 | 2,523,611 | 37,874 | 839,445 |
| | | | (74%) | (1%) | (25%) |
| Buccal Sample 2 | Yes | 3,896,003 | 1,581,395 | 112,202 | 2,202,406 |
| | | | (41%) | (3%) | (57%) |
| Nasopharyngeal | Yes | 3,196,086 | 1,970,031 | 173,974 | 1,052,081 |
| carcinoma slide | | | | (5%) | (33%) |

### Example 2

### Application to Linearly Amplified DNA

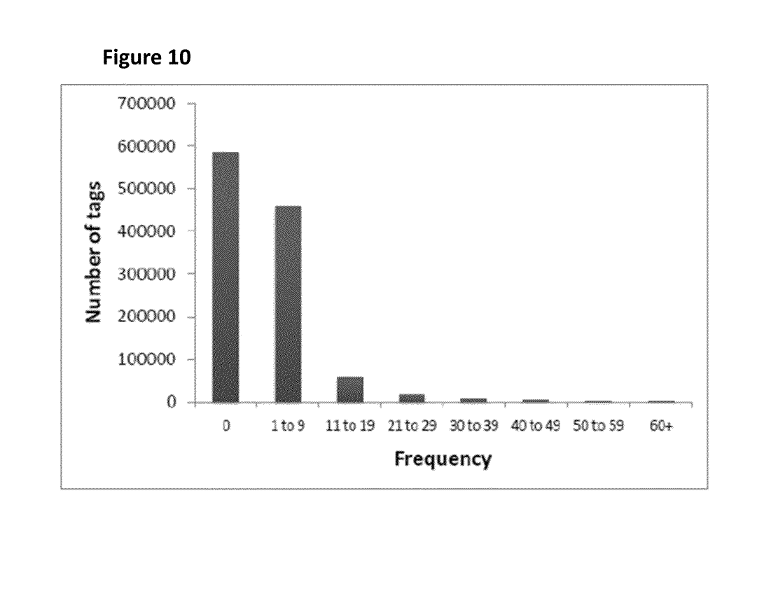

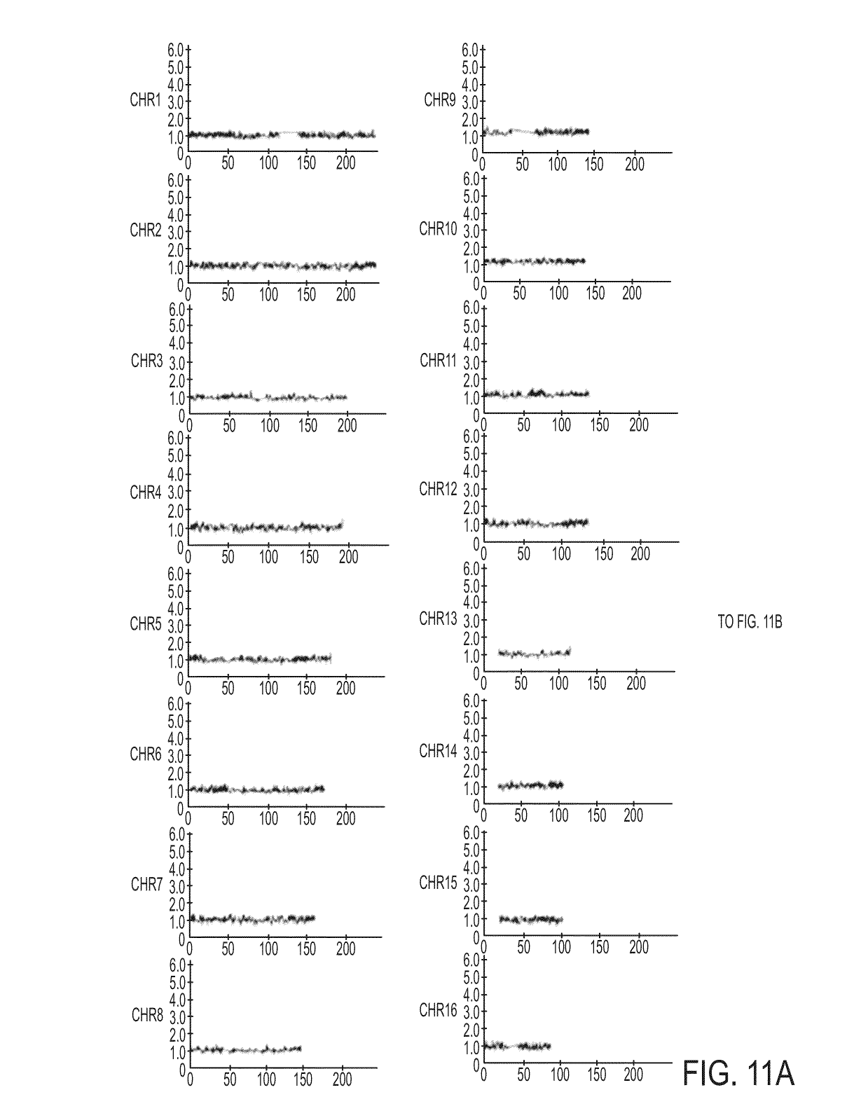

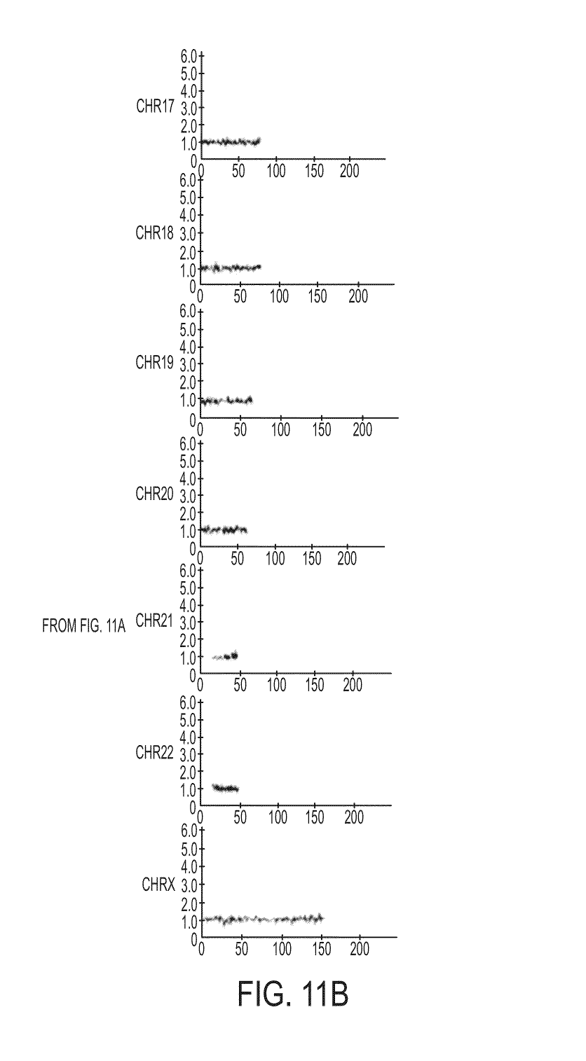

To demonstrate that the methods of the invention could be used effectively with small amounts of DNA amplified by linear, multiple displacement (phi29) amplification, 1 ng of the blood-derived human genomic DNA was amplified to yield 1 ug of total material. 4,091,327 tags were recovered from amplified material, of which 3,868,735 (95%) were perfect matches for human sequence (Table 2). 50.0% of all human tags were recovered. Comparison of the human karyotype of amplified an unamplified DNA demonstrated a high degree of linearity of the amplified material, although tag recovery was not as perfectly linear as with unamplified material (FIG. 8). Regression analysis revealed very high correlation coefficients for observed vs. expected tag counts per chromosome (r2=0.976 for amplified material). The distribution of recovered single copy tags did not reveal significant skewing relative to analysis of non-amplified material (FIG. 10). Karyotype analysis of amplified material showed no artifactual amplifications or deletions (FIG. 11). No microbial sequences were recovered. Three tags were recovered for human endogenous retrovirus H. These results demonstrate that genomic DNA samples as small as 1 ng can be effectively analyzed with near-quantitative recovery of tags using the methods of the invention.

Table 2 shows expected and recovered BsaXI tags per human chromosome from human blood sample by the method of the invention

| | | Obtained sequence | |
|---|---|---|---|
| Chromosome | BsaXI sites | tags | Fold coverage |
| 1 | 87,161 | 804,023 | 9.225 |
| 2 | 84,481 | 766,541 | 9.074 |
| 3 | 67,034 | 608,038 | 9.071 |
| 4 | 56,483 | 493,753 | 8.742 |
| 5 | 59,462 | 531,790 | 8.943 |
| 6 | 57,599 | 513,989 | 8.924 |
| 7 | 53,411 | 482,168 | 9.028 |
| 8 | 50,748 | 458,119 | 9.027 |
| 9 | 41,938 | 377,088 | 8.992 |
| 10 | 49,724 | 449,742 | 9.045 |
| 11 | 51,136 | 466,689 | 9.126 |
| 12 | 47,363 | 428,804 | 9.054 |
| 13 | 30,671 | 276,701 | 9.022 |
| 14 | 32,461 | 295,323 | 9.098 |
| 15 | 30,618 | 280,307 | 9.155 |
| 16 | 32,319 | 300,618 | 9.302 |
| 17 | 34,930 | 325,020 | 9.305 |
| 18 | 26,405 | 238,530 | 9.034 |
| 19 | 27,487 | 256,823 | 9.343 |
| 20 | 27,566 | 258,565 | 9.380 |
| 21 | 12,295 | 111,352 | 9.057 |
| 22 | 17,444 | 166,189 | 9.527 |
| X | 42,375 | 194,271 | 4.585 |
| Y | 1,985 | 9,395 | 4.733 |

### Example 3

### Application to Biome Characterization

The sensitivity of the methods of the invention for detection of non-human DNA was tested by spiking a human blood sample with purified E. coli genomic DNA. 1 ug of human blood DNA was combined with 20 pg of E. coli DNA (1:50,000 by weight, ˜1% by molar genome). As this sample was analyzed in multiplex (using a 2 bp barcode embedded in the adaptor), fewer total tags were recovered. Of the 681,325 tags recovered, 2,104 (0.3%) were found to be perfect matches for E. coli. Four hundred sixty four of the 988 potential distinct E. coli sequence tags were recovered. No other tags meeting criteria for any other microbial genome were identified.

The biome of the oral mucosa was identified and characterized using the methods of the invention to determine its ability to identify the organisms found in a complex host microbial environment. DNA was obtained from buccal brushings of two individuals and amplified with phi29 methodology. The first sample yielded 3,400,930 sequence tags, of which 2,523,611 (74%) were human (Table 1). 37,874 (1%) tags were perfect matches for the microbial database while 839,445 (25%) matched neither human sequence nor known microbial or viral sequence. In the second sample, 3,896,003 tags were recovered, of which 1,581,395 (41%) were of human origin (Table 1). 112,202 tags (3%) were perfect matches for microbial or viral sequences. 2,202,406 (57%) sequences matched neither human nor microbial/viral databases. Human karyotypes for both samples were highly linear indicating quantitative recovery of human DNA. A microbial species was considered identified when two or more tags unique in the database to that species were recovered in an individual's buccal mucosa sample. None of the putative microbial matches were found in analysis of blood, HEK 293, SW480, or HT-29 human cell lines, indicating that these are bona fide microbial sequences and not contaminant sequences or sequences shared between human and microbial genomes. Organisms corresponding to recovered tags found in both individuals' oral mucosa are shown in Table 3.

Table 3 shows identities of microbial sequences identified by analysis of two buccal swab samples

| | Sample 1 | Sample 2 | Found in | Found in |
|---|---|---|---|---|
| | unique | unique | Nasidze | Keijser |
| Organism | score | score | et al. | et al. |
| Streptococcus mitis | 22.55 | 43.12 | X | X |
| Streptococcus | 21.61 | 42.15 | X | X |
| pneumoniae | | | | |
| Streptococcus sanguinis | 3.49 | 3.70 | X | X |
| Veillonella parvula | 22.53 | 3.42 | X | X |
| Fusobacterium | 9.46 | 1.98 | X | X |
| nucleatum | | | | |
| Streptococcus gordonii | 3.63 | 1.31 | X | X |
| Haemophilus influenzae | 0.18 | 1.00 | X | X |
| Aggregatibacter | 0.12 | 0.85 | | X |
| aphrophdus | | | | |
| Rothia mucilaginosa | 0.12 | 0.84 | X | X |
| Haemophilus somnus | 0.20 | 0.39 | X | X |
| Leptotrichia buccalis | 2.29 | 0.36 | X | X |
| Streptococcus agalactiae | 0.04 | 021 | X | X |
| Streptococcus oralis | 0.19 | 0.18 | X | X |
| Neisseria meningitidis | 0.13 | 0.07 | X | X |
| Capnocytophaga | 3.75 | 0.07 | X | X |
| ochracea | | | | |
| Streptococcus | 0.01 | 0.03 | X | X |
| dysgalactiae | | | | |
| Streptococcus | 0.04 | 0.02 | X | X |
| thermophilus | | | | |
| Actinobacillus | 0.01 | 0.02 | X | X |
| pleuropneumoniae | | | | |
| Atopobium parvulum | 0.88 | 0.02 | | |
| Porphyromonas | 1.87 | 0.02 | X | X |
| gingivalis | | | | |
| Bacteroides fragilis | 0.07 | 0.02 | | X |
| Treponema denticola | 0.10 | 0.01 | X | X |
| Campylobacter concisus | 0.03 | 0.01 | X | X |
| Fusobacterium | 0.01 | 0.01 | X | X |
| periodonticum | | | | |
| Bacteroides | 0.04 | 0.01 | | X |
| thetaiotaomicron | | | | |
| Clostridium difficile | 0.19 | 0.01 | X | X |
| Enterococcusfaecalis | 0.03 | 0.00 | X | |
| Granulicatella adiacens | 0.01 | 0.00 | X | |
| Streptobacillus | 0.05 | 0.00 | X | X |
| moniliformis | | | | |
| Streptococcus | 6.11 | | X | X |
| parasanguinis | | | | |
| Aggregatibacter | 0.26 | | X | X |
| actinomycetemcomitans | | | | |
| Streptococcus | 0.01 | | X | X |
| vestibularis | | | | |
| Prevotella nigrescens | | 0.05 | X | X |
| Clostridiales genomo sp. | | 0.02 | | |
| Lactobacillus salivarius | | 0.01 | X | X |
| Streptococcus equi | | 0.01 | X | X |
| Lactobacillus fermentum | | 0.00 | X | X |

A total of 29 species were identified in common from both patients' samples. Sequences from Streptococcus species were the most commonly recovered and accounted for 57.5% and 90.7% of all microbial tags recovered in the individual samples, respectively. 18 genera in total were identified. All have been previously identified in large-scale, deep sequencing of 16S DNA of the oral mucosa (Keijser et al. 2008; Nasidze et al. 2009a; Nasidze et al. 2009b; Zaura et al. 2009). While the majority of species were found in both individuals' samples, significant differences in quantitative recovery were found. In particular, Veillonella parvula, a gram-negative, anaerobic bacterium found as commensal in multiple human mucosal sites, accounted for 22.5% of tags in the first sample, but only 3.4% of tags in the second. A total of eight species were detected in only one individual's saliva, the most prevalent being Streptococcus parasanguinis which constituted 6.1% of recovered tags from the first subject's sample but was not found in the second subject.

In both samples, the majority of apparent non-human tags were not found in the NCBI database (25% and 57% of total tags, respectively). Twenty of most abundantly recovered unknown sequence tags found in saliva were selected of one individual but not blood or cell line DNA for further analysis. Using the vectorette genomic DNA walking technique, additional genomic sequences were generated ranging from 298 to 991 bp from eight of these sequence tags. Analysis against the NCBI database revealed that all but one tag were unique and novel sequences in the non-redundant DNA database. These sequences were termed Genome Unknown Sequences (GUS). The eighth tag was found to be from a human gene sequence identified in a genome build subsequent to the build utilized in the bioinformatics software. To identify possible organisms accounting for these sequences, a translated BLAST search was performed for each sequence. While only GUS 3 was a near-perfect match (for Haemophilus influenza), five of the six remaining GUS tags yielded high probability matches (Table 4).

Table 4 shows translated BLAST matches for prevalent GUS sequences

| GUS# | Protein | Organism | Frame | ID | Positive | E-value |
|---|---|---|---|---|---|---|
| 1 | Hypothetical protein | Capnocytophaga ochracea | −1 | 63% | 74% | 2 × 10⁻²³ |
| | CochFRAFT_04770 | DSM 7271 | | | | |
| 2 | Asparagine synthetase | Clostridium botulinum | −1 | 61% | 77% | 1 × 10⁻¹⁸ |
| | AsnA | F str. Langeland | | | | |
| 3 | COG0468: RecA/RadA | Haemophilus influenzae | −3 | 95% | 98% | 2 × 10⁻¹¹⁸ |
| | recombinase | R2866 | | | | |
| 4 | Hypothetical protein | Streptococcus pyogenes | +2 | 69% | 81% | 2 × 10⁻²⁴ |
| | SpyM3_0722 | phage MGAS315 | | | | |
| 5 | Transcription regulator | Streptococcus gordonii | −3 | 69% | 83% | 3 × 10⁻⁴⁷ |
| | | str. Challis substr. CH1 | | | | |
| 6 | No match | | | | | |
| 7 | Terminal protein | Actinomyces phage Av-1 | +2 | 30% | 54% | 5 × 10⁻¹⁴ |

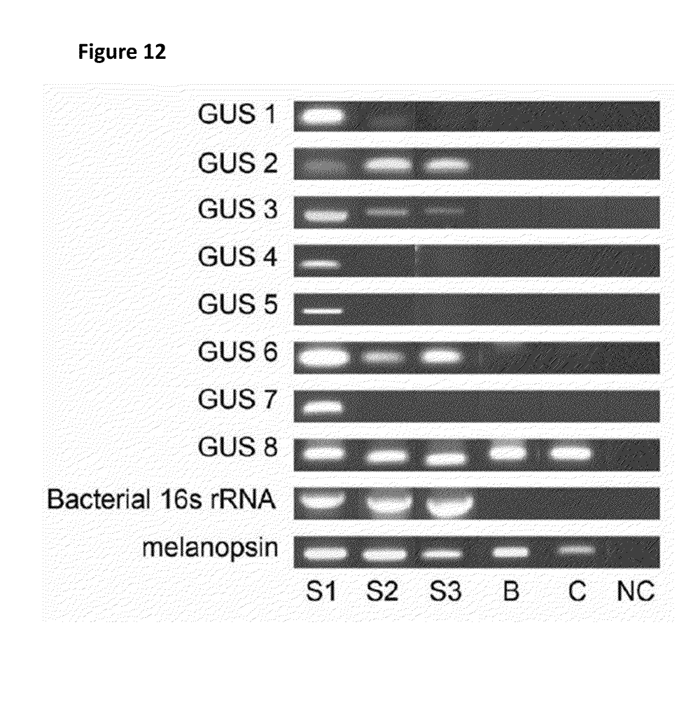

All were homologous to microbially-derived sequences, including two phage sequences (GUS 4 for a Streptococcus pyogenes phage (E value 2×10⁻²⁴) and GUS 7 for an Actinomyces phage (E value 5×10⁻¹⁴). Unique PCR primers were generated for the novel sequences, targeting sequences outside the original BsaXI tag. As shown in FIG. 12, three tag sequences (GUS 2, 3, and 6) were found in saliva of all individuals but not found in blood or HEK293 cell line DNA. The remaining three GUS tags appeared unique to the individual in whom they were identified.

### Example 4

### Application to Pathogen Detection

An attractive feature of digital karyotyping in pathogen detection and discovery is the ability to find potential pathogens associated with specific disease conditions. Most cases of nasopharyngeal carcinoma are associated with Epstein-Barr virus (EBV, HHV-4), which is thought to be causative of disease. To determine if the methods of the invention has adequate sensitivity to detect a virally-mediated carcinoma, two fixed, paraffin-embedded microscope slides of a nasopharyngeal carcinoma specimen were subjected to the method following phi29 amplification of recovered DNA. A total of 1,970,031 human sequences were recovered. 81,799 tags (4.1%) were recovered that were perfect matches for HHV-4. Additionally, 16, 826 tags were recovered that were perfect matches for either Delftia acidovorans, Stenotrophomonas maltophilia, Propionibacterium acnes, or Cupravidus metalidurans. It is assumed that the latter were bacterial contaminants found on the surface of the pathology specimen slides.

### Example 5

### Application to Measuring Mitochondrial Density

An additional feature of digital karyotyping in disease detection and discovery is the ability to find potential sequence tags associated with specific disease conditions. For example sequence tags attributable to human mitochondrial sequences can be quantified and compared with human chromosomal genomic tags, to yield a measure of mitochondrial density in the tissue analyzed. A count of the number of tags attributable to mitochondria and divided by the mean representation of human genomic sequence tags will provide of ratio of mitochondria/nucleus or similar. This application would be useful in detecting or diagnosing diseases related to mitochondrial density or dysfunction (non-limiting examples include; muscle disorders, metabolic disorders, Type 2 diabetes, Parkinson's disease, atherosclerotic heart disease, stroke, Alzheimer's disease, and cancer).

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. Accordingly, the disclosure is not limited.

We claim:

1. A method for identifying a DNA sequence, the method comprising:
receiving sequence-tag data that indicates a first set of sequence tags, wherein each sequence tag in the first set of sequence tags is associated with a cutting of a nucleic acid sequence by a Type IIB DNA restriction enzyme, wherein the nucleic acid sequence is associated with one or more unidentified organisms represented in a sample;
comparing a first sequence tag in the first set of sequence tags to each sequence tag in a second set of sequence tags, wherein each sequence tag in the second set of sequence tags is associated with a portion of one of a plurality of nucleic acid sequences, the portion identified based on the Type IIB DNA restriction enzyme, and wherein each nucleic acid sequence in the plurality of nucleic acid sequences is associated with a one or more identified organisms;
determining identification data that indicates a potential identity of at least one of the one or more identified organisms based on a match between the first sequence tag and a second sequence tag in the second set of sequence tags; and
causing a graphical display to provide a visual representation of the identification data.

2. The method of claim 1, wherein the second set of sequence tags is stored in a database, the database further comprising metadata associated with each sequence tag in the second set of sequence tags, wherein metadata associated with the second sequence tag indicates that the second sequence tag is associated with a particular organism of the one or more identified organisms, and wherein the identification data indicates that the potential identity of the at least one of the one or more unidentified organisms comprises the particular organism.

3. The method of claim 2, wherein the second sequence tag is present only once in a full genome of the particular organism, and wherein metadata associated with the second sequence tag indicates (i) a genomic location of the second sequence tag and (ii) that the second sequence tag is present only once in the full genome of the particular organism.

4. The method of claim 2, wherein the second sequence tag is associated with only the particular organism and is present two or more times within a genome of the particular organism, and wherein metadata associated with the second sequence tag indicates that the second sequence tag is a potential identifier of the particular organism.

5. The method of claim 2, wherein the second sequence tag is associated with a group of organisms in the one or more identified organisms, wherein the group of organisms includes the particular organism, and wherein the metadata associated with the second sequence tag indicates that the second sequence tag is a potential identifier of each organism in the group of organisms, and wherein the identification data indicates that the potential identity of the one or more unidentified organisms comprises the organisms in the group of organisms.

6. The method of claim 5, wherein the group of organisms is one of bacteria, fungi, parasites, viruses, phage, vertebrates, or invertebrates.

7. The method of claim 2, wherein the identification data further indicates a percentage of representation by the particular organism in the sample.

8. The method of claim 1, wherein each sequence tag in the first set of sequence tags has a length between 20-33 nucleotides.

9. The method of claim 1, wherein each sequence tag in the second set of sequence tags has a length between 20-33 nucleotides.

10. The method of claim 1, wherein the Type IIB DNA restriction enzyme is selected from the group consisting of AjuI, AlfI, AloI, ArsI, BaeI, BarI, BcgI, BdaI, BplI, BsaXI, Bsp24I, CjeI, CjePI, CspCI, FalI, HaeI, Hin4I, NgoAVIII, NmeDI, PpiI, PsrI, RdeGBIII, SdeOSI, TstI and UcoMSI.

11. The method of claim 10, wherein the Type IIB DNA restriction enzyme is BsaXI.

12. A method of biome representational in silico karyotyping a sample, comprising:
(a) extracting genomic DNA from the sample;
(b) cutting the genomic DNA in the sample with a Type IIB DNA restriction enzyme to create a set of DNA restriction fragments;
(c) ligating oligonucleotide adaptors to the set of DNA restriction fragments;
(d) separating the oligonucleotide adaptors ligated to the set of DNA restriction fragments to isolate the set of DNA restriction fragments;
(e) sequencing the set of DNA restriction fragments to generate a first set of sequence tags;
(f) identifying the first set of sequence tags according the method of claim 1.

13. The method of claim 12, wherein the oligonucleotide adaptors comprise a nucleic acid structure represented by formula [I]:
5′-L-X-M-N-3′ [I]
wherein:
L is an optional 5′ label;
X is a nucleotide sequence complementary to solid-phase bridge oligonucleotides;
M is an optional nucleotide barcode; and
N is a nucleotide that comprises a sequence capable of hybridizing with a two or three nucleotide 3′ overhang of the Type IIB DNA restriction enzyme.

14. The method of claim 13 wherein L is present in the oligonucleotide adaptors and is selected from the group consisting of biotin, poly-histidine, Myc, FLAG, HA, glutathione-S-transferase or a magnetic bead.

15. The method of claim 13 wherein X is selected from the group consisting of
(SEQ ID NO: 1)
5′-AATGATACGGCGACCACCGAGATCTACACTCTTTCCCTACACGACGCTCTTCCGATCT-3′;
(SEQ ID NO: 2)
5′-AGATCGGAACAGCGTCGTGTAGGGAAAGAGTGTAGATCTCGGTGGTCGCCGTATCATT-3′;
(SEQ ID NO: 3)
5′-CAAGCAGAAGACGGCATACGAGCTCTTCCGATC-3′;
and
(SEQ ID NO: 4)
5′-GATCGGAAGAGCTCGATATCCGTCTTCTGCTTG-3′.

16. The method of claim 13, wherein the method comprises multiplex sequencing, and wherein M is present in the oligonucleotide adaptors and comprises two, three or four nucleotides.

17. The method of claim 16, wherein the nucleotide barcode M of the oligonucleotide adaptors is at least two Levenshtein edit distances apart from the nucleotide barcode of the oligonucleotide adaptors for a second sample.

18. An oligonucleotide adaptor comprising a nucleic acid structure represented by formula [I]:
5′-L-X-M-N-3′ [I]
wherein:
L is an optional 5′ label;
X is a nucleotide sequence complementary to solid-phase bridge oligonucleotides;
M is an optional nucleotide barcode; and
N is a nucleotide that comprises a sequence capable of hybridizing with a two or three nucleotide 3′ overhang of the Type IIB DNA restriction enzyme.

19. The oligonucleotide adaptor of claim 18 wherein T is present in the oligonucleotide adaptors and is selected from the group consisting of biotin, poly-histidine, Myc, FLAG, HA, glutathione-S-transferase or a magnetic bead.

20. The oligonucleotide adaptor of claim 18 wherein X is selected from the group consisting of
(SEQ ID NO: 1)
5′-AATGATACGGCGACCACCGAGATCTACACTCTTTCCCTACACGACGCTCTTCCGATCT-3′;
(SEQ ID NO: 2)
5′-AGATCGGAACAGCGTCGTGTAGGGAAAGAGTGTAGATCTCGGTGGTCGCCGTATCATT-3′;
(SEQ ID NO: 3)
5′-CAAGCAGAAGACGGCATACGAGCTCTTCCGATC-3′;;
and
(SEQ ID NO: 4)
5′-GATCGGAAGAGCTCGATATCCGTCTTCTGCTTG-3′.

21. The oligonucleotide adaptor of claim 18, wherein M is present in the oligonucleotide adaptors and comprises a nucleotide barcode comprising two, three or four nucleotides.
